(19) United States
(12) Patent Application Publication
(10) Pub. No.: US 2018/0049639 A1
(43) Pub. Date: Feb. 22, 2018

(54) DRY ELECTRODE, ITS MANUFACTURING METHOD AND BIO-ELECTROMAGNETIC WAVE DETECTING DEVICE AND SENSOR ELEMENT COMPRISING THE DRY ELECTRODE

(71) Applicant: BOE TECHNOLOGY GROUP CO., LTD., Beijing (CN)

(72) Inventor: Xueyan TIAN, Beijing (CN)

(73) Assignee: BOE TECHNOLOGY GROUP CO., LTD., Beijing (CN)

(21) Appl. No.: 15/651,038

(22) Filed: Jul. 17, 2017

(30) Foreign Application Priority Data
Aug. 19, 2016 (CN) 201610697261.2

(51) Int. Cl. A61B 5/00; A61B 5/0408; A61B 5/0478; A61N 1/04; A61B 5/024

(52) U.S. Cl. CPC A61B 5/0006; A61B 5/6833; A61B 5/02444; A61B 5/0478; A61N 1/04; A61B 5/04085

(57) ABSTRACT

The present disclosure provides a dry electrode for a bio-electromagnetic wave detecting device, its manufacturing method, and a sensor element and a bio-electromagnetic wave detecting device comprising the dry electrode. The dry electrode comprises: a flexible substrate, at least one set of protruding structures arranged on the flexible substrate, electrode lead-out terminals and electrode lead-out wires, wherein the protruding structure comprises an inner core made of a flexible insulating material, and a conductive thin film coated on an outer side of the inner core.

### CROSS-REFERENCE TO RELATED APPLICATION

The present application claims a priority to the Chinese Patent Application No. 201610697261.2 filed on Aug. 19, 2016, the disclosures of which are incorporated herein in its entirety by reference.

### TECHNICAL FIELD

The present disclosure relates to the field of health monitoring technology, in particular to a dry electrode for a bio-electromagnetic wave detecting device, its manufacturing method and a bio-electromagnetic wave detecting device and a sensor element comprising the dry electrode.

### BACKGROUND

In traditional detection of electroencephalogram (EEG) or electrocardiogram (ECG), hospitals would adopt wet electrodes (Ag/AgCl). Taking brainwave detection as an example, a patient will be required to put on a complex test hat to which several electrodes (Ag/AgCl) are fixed in accordance with the international 10-20 system, and after the patient has put on the test hat, the electrodes are attached to the scalp. Then, lead wires are connected and each of the electrodes is filled with a conductive fluid sol. In this case, a primary cell is formed due to a chemical reaction taking place between ions in the conductive fluid sol and the electrode, which makes the contact resistance very low. After confirming that each contact is in full contact with the scalp, the detection may begin. As can thus be seen, the traditional detection method using the wet electrodes comprises many steps and the process is relatively complex; since washing is required after the detection, the traditional method takes a long time; moreover, if there are more test electrodes, factors that affect the accuracy of the detection result will also increase.

In order to overcome the shortcomings of the traditional wet electrodes, a detection method using dry electrodes was developed. The dry electrodes are usually made of a metal, such as silver. Contact type silver electrodes are prepared on a substrate and used to make contact with the brain for testing, which avoids the inconvenience and other issues caused by the use of wet electrodes. However, as compared with the wet electrodes, the dry electrodes produce large contact resistance, thereby leading to high interference, low accuracy of the obtained detection data and other issues.

### SUMMARY

The present disclosure provides a dry electrode for a bio-electromagnetic wave detecting device, its manufacturing method and a bio-electromagnetic wave detecting device and a sensor element comprising the dry electrode, so as to solve the problem existing in the prior art that large contact resistance is generated due to the use of dry electrodes.

According to one aspect, the present disclosure provides a dry electrode for a bio-electromagnetic wave detecting device, comprising: a flexible substrate, at least one set of protruding structures arranged on the flexible substrate, an electrode lead-out terminal for electrically interconnecting the protruding structures in each set of the protruding structures, and electrode lead-out wires electrically connected to the electrode lead-out terminals in a one-to-one corresponding manner, wherein the protruding structure comprises an inner core made of a flexible insulating material and a conductive thin film coated on an outer side of the inner core.

In an optional embodiment, the conductive thin film, the electrode lead-out terminals and the electrode lead-out wires are made of a metallic carbon nanotube material.

In an optional embodiment, the flexible substrate and the inner cores are made of polydimethylsiloxane.

In an optional embodiment, the inner core and the flexible substrate are an integrally-formed structure.

In an optional embodiment, the inner cores are fixed to the flexible substrate using a conductive adhesive.

According to another aspect, the present disclosure further provides a method of manufacturing the dry electrode for a bio-electromagnetic wave detecting device as described above, comprising steps of:

providing a flexible substrate and inner cores arranged on the flexible substrate;

forming protruding structures by coating a conductive thin film on an outer side of each of the inner cores; and

forming, on each set of the protruding structures, an electrode lead-out terminal for electrically interconnecting the protruding structures, and electrode lead-out wires electrically connected to the electrode lead-out terminals in a one-to-one corresponding manner.

In an optional embodiment, the flexible substrate and the inner cores arranged on the flexible substrate are formed integrally.

In an optional embodiment, the step of integrally forming the flexible substrate and the inner cores arranged on the flexible substrate comprises:

placing a mixed solution of polydimethylsiloxane and a hardening agent in a mold and subjecting it to a curing treatment;

forming an integrally-formed structure of the inner cores and the flexible substrate after the completion of the curing treatment; and

performing a mold release treatment.

In an optional embodiment, the step of forming the protruding structures by coating the conductive thin film on the outer side of each of the inner cores comprises:

coating a solution of metallic carbon nanotubes on the outer side of each of the inner cores to form the conductive thin film using a dip-coating method, thereby forming the protruding structures.

In an optional embodiment, prior to coating the solution of the metallic carbon nanotubes on the outer side of each of the inner cores using the dip-coating method, the manufacturing method further comprises subjecting the solution of the metallic carbon nanotubes to acid treatment.

According to another aspect, the present disclosure provides a method of manufacturing the dry electrode for a bio-electromagnetic wave detecting device as described above, comprising steps of:

forming a plurality of inner cores using a flexible insulating material and forming protruding structures by coating a conductive thin film on an outer side of each of the inner cores;

fixing each of the protruding structures to a flexible substrate using a conductive adhesive; and

forming, on each set of the protruding structures, an electrode lead-out terminal for electrically interconnecting the protruding structures, and electrode lead-out wires electrically connected to the electrode lead-out terminals in a one-to-one corresponding manner.

In an optional embodiment, the step of forming the plurality of the inner cores using the flexible insulating material and forming the protruding structures by coating the conductive thin film on the outer side of each of the inner cores comprises:

forming the plurality of the inner cores using polydimethylsiloxane; and

immersing each of the inner cores in a solution of metallic carbon nanotubes for a period of time to form the conductive thin film coated on the outer side of each of the inner cores, thereby forming the protruding structures.

In an optional embodiment, prior to immersing each of the inner cores in the solution of metallic carbon nanotubes for a period of time, the method further comprises subjecting the solution of the metallic carbon nanotubes to acid treatment.

According to a further aspect, the present disclosure further provides a sensor element for a bio-electromagnetic wave detecting device comprising a working electrode for detecting a potential at a position to be detected in an organism and a reference electrode.

According to a further aspect, the present disclosure further provides a bio-electromagnetic wave detecting device comprising: a working electrode for detecting a potential at a position to be detected in an organism and a reference electrode; a preamplifier connected to both the working electrode and the reference electrode; a processor connected to the preamplifier and an output module connected to the processor,

wherein the working electrode and/or the reference electrode are the dry electrode for a bio-electromagnetic wave detecting device as described above.

In an optional embodiment, the bio-electromagnetic wave detecting device is a brainwave detecting device or an electrocardiogram detecting device.

At least one of the embodiments according to the present disclosure produce the following advantageous effects:

In the dry electrode according to the embodiments of the present disclosure, the protruding structure comprises the inner core made of the flexible insulating material and the conductive thin film coated on the outer side of the inner core. Thus, when the dry electrode is in contact with the skin, a small contact resistance is generated, thus making it possible to reduce signal interference and increase the sensitivity of the sensor element and the accuracy of the detection data of the bio-electromagnetic wave detecting device. Moreover, since both the flexible substrate and the inner cores are made of the flexible material, and the conductive thin film coated on the outer side of the inner core is very thin, the dry electrode is flexible and then when the dry electrode is in contact with the skin, no discomfort sensation will be felt even in close contact, and in other word, the comfort is increased during the detection of the bio-electromagnetic wave.

### BRIEF DESCRIPTION OF THE DRAWINGS

FIG. 1 is a structural schematic view of a dry electrode for a bio-electromagnetic wave detecting device according to an embodiment of the present disclosure;

FIG. 2 is a flow chart of a method of manufacturing the dry electrode for a bio-electromagnetic wave detecting device according to an embodiment of the present disclosure;

FIG. 3 is a flow chart of a method of manufacturing the dry electrode for a bio-electromagnetic wave detecting device according to another embodiment of the present disclosure; and

FIG. 4 is a structural schematic view of a bio-electromagnetic wave detecting device according to an embodiment of the present disclosure.

### DETAILED DESCRIPTION

To solve the problem existing in the prior art that large contact resistance is generated due to the use of a dry electrode, the present disclosure provides in some embodiments a dry electrode for a bio-electromagnetic wave detecting device, its manufacturing method and a bio-electromagnetic wave detecting device and a sensor element comprising the dry electrode.

Hereinafter, specific embodiments of the dry electrode for a bio-electromagnetic wave detecting device, its manufacturing method and the bio-electromagnetic wave detecting device and a sensor element according to the present disclosure will be illustrated in detail with reference to the accompanying drawings. The sizes and shapes of the structures in the drawings do not reflect the true proportions, and are merely for illustrative purpose.

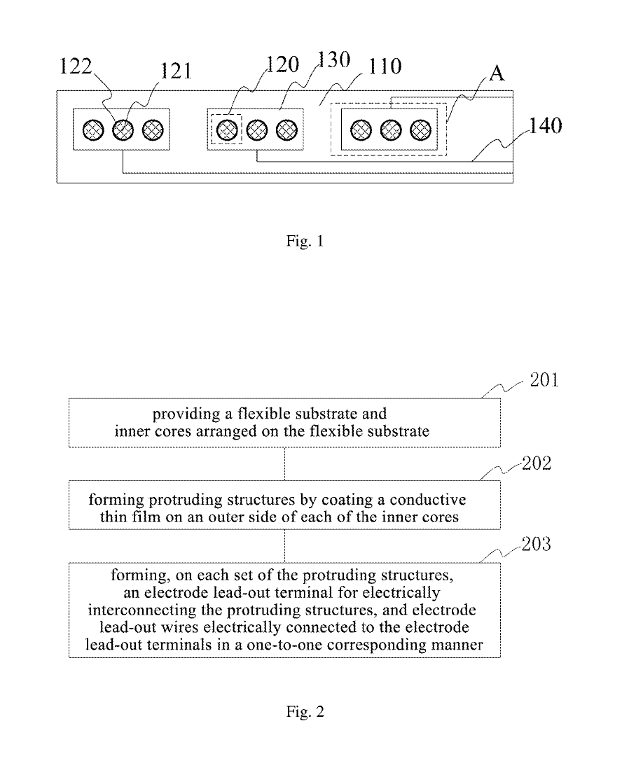

As shown in FIG. 1, an embodiment of the present disclosure provides a dry electrode for a bio-electromagnetic wave detecting device, comprising: a flexible substrate 110; at least one set of protruding structures 120 arranged on the flexible substrate 110; an electrode lead-out terminal 130 for electrically interconnecting the protruding structures 120 in each set of the protruding structures; and electrode lead-out wires 140 electrically connected to the electrode lead-out terminals 130 in a one-to-one corresponding manner; wherein the protruding structure 120 comprises an inner core 121 made of a flexible insulating material, and a conductive thin film 122 coated on an outer side of the inner core 121.

In the dry electrode provided in this embodiment of the present disclosure, the protruding structure 120 comprises the inner core 121 made of the flexible insulating material, and the conductive thin film 122 coated on the outer side of the inner core 121. Thus, when the dry electrode is in contact with the skin, a small contact resistance is generated, thus making it possible to reduce signal interference and increase the accuracy of the detection data of the bio-electromagnetic wave detecting device. Moreover, since both the flexible substrate 110 and the inner cores 121 are made of the flexible material and the conductive thin film 122 coated on the outer side of the inner core 121 is very thin, the dry electrode is flexible and when the dry electrode is in contact with the skin, no discomfort sensation will be felt even in close contact, and in other word, the comfort is increased during the detection of the bio-electromagnetic wave. The dry electrode may be applied to various bio-electromagnetic wave detecting devices, for example, a brainwave detecting device or an electrocardiogram detecting device. Further, it may also be applied to non-medical fields such as commercial brainwave toy products, for example brainwave smart wearable products.

In the dry electrode provided in this embodiment of the present disclosure, the flexible substrate 110 is provided with at least one set of protruding structures 120, and this can ensure that when performing the detection of bio-electromagnetic wave, the dry electrode is in close contact with the skin or scalp to maintain good electrical contact, thereby increasing the accuracy of the detection data. In FIG. 1, the protruding structure 120 is shown having a spherical shape (a circular shape in a top view), which is a preferred shape in the embodiment of the present disclosure. In an optional embodiment, the protruding structure 120 may have any other shapes. As shown in FIG. 1, the dotted frame A represents a set of the protruding structures 120. FIG. 1 schematically show that each dry electrode includes three sets of protruding structures 120 and each of the sets includes three protruding structures 120. The shape and number of the protruding structures 120 are not limited thereto.

In the dry electrode provided in the optional embodiment of the present disclosure, the conductive thin film 122, the electrode lead-out terminals 130 and the electrode lead-out wires 140 are made of a metallic carbon nanotube material.

The conductive thin film 122 made of the metallic carbon nanotube (CNT) material has good electrical conductivity and a small thickness. It is thus preferable to make the conductive thin film 122 using the metallic carbon nanotube material. The conductive thin film 122 may be also made of any other conductive materials, and its material is not defined here.

In an optional embodiment, the electrode lead-out terminals 130 and the electrode lead-out wires 140 are made of the metallic carbon nanotube material. When the dry electrode is applied to various bio-electromagnetic wave detecting devices, the electrode lead-out terminals 130 and the electrode lead-out wires 140 may electrically connect the protruding structures 120 to the bio-electromagnetic wave detecting device, so it is able to input a signal collected by the dry electrode into the bio-electromagnetic wave detecting device. Since the metallic carbon nanotube material has good electrical conductivity, the electrode lead-out terminals 130 and the electrode lead-out wires 140 made of the metallic carbon nanotube material may ensure good contact and smooth transmission of signals. The electrode lead-out terminals 130 and the electrode lead-out wires 140 may be made of any other conductive material, and the material is not limited thereto.

In the dry electrode provided in an optional embodiment of the present disclosure, the flexible substrate 110 and the inner cores 121 are preferably made of polydimethylsiloxane (PDMS).

In the dry electrode provided in the embodiment of the present disclosure, both the flexible substrate 110 and the inner core 121 are designed to be made of a flexible material, thus making it possible to allow the dry electrode to have good biocompatibility. Since the inner core 121 is designed to be made of the flexible material, the protruding structure 120 is relatively soft. When the protruding structure 120 is in contact with the skin or scalp, no discomfort sensation will be felt even in close contact; moreover, when the dry electrode in which the flexible substrate 110 is made of the flexible material is applied to various bio-electromagnetic wave detecting devices, the dry electrode can be designed into various shapes as desired so as to adapt to the detection of a human body or animal and increase the comfort during the detection. Moreover, the bio-electromagnetic wave detecting device comprising the dry electrode may be made into a flexible wearable device for real-time detection of the bio-electromagnetic wave.

The flexible substrate 110 and the inner cores 121 may be also made of different materials so long as each of them is made of a flexible material.

It is preferable that both the flexible substrate 110 and the inner cores 121 are made of PDMS, but the material of them is not limited thereto.

In the dry electrode provided according to the embodiment of the present disclosure, the inner cores 121 and the flexible substrate 110 may be an integrally-formed structure. When the inner cores 121 and the flexible substrate 110 are made of the same material, they may be integrally formed simultaneously, thereby economizing the process steps and saving the cost.

In the dry electrode provided according to the embodiment of the present disclosure, the inner cores 121 may be fixed to the flexible substrate 110 using a conductive adhesive. The separate preparation of the inner cores 121 and the flexible substrate 110 may make a subsequent step of coating a conductive thin film 122 on the outer side of the inner core 121 simpler.

The dry electrode for the bio-electromagnetic wave detecting device provided in the embodiments according to the present disclosure may be manufactured using the following two methods:

Method 1:

Based on the same inventive concept, this embodiment of the present disclosure provides a method of manufacturing the above-mentioned dry electrode for a bio-electromagnetic wave detecting device. As shown in FIG. 2, the manufacturing method comprises the following steps:

S201: integrally forming a flexible substrate and inner cores arranged on the flexible substrate;

S202: forming protruding structures by coating a conductive thin film on an outer side of each of the inner cores;

S203: forming, on each set of the protruding structures, an electrode lead-out terminal for electrically interconnecting the protruding structures, and electrode lead-out wires electrically connected to the electrode lead-out terminals in a one-to-one corresponding manner.

Since both the flexible substrate and the inner cores may be made of PDMS, the flexible substrate and the inner cores may be integrally formed. This avoids a complex process in which the flexible substrate and inner cores are separately prepared and then the inner cores are fixed to the flexible substrate, and thus economizes two steps and saves the cost.

In the manufacturing method provided in the embodiment of the present disclosure, the step S201 may include (not shown in the figures):

S2011: placing a mixed solution of polydimethylsiloxane and a hardening agent in a mold and subjecting it to a curing treatment;

S2012: forming an integrally-formed structure of the inner cores and the flexible substrate after the completion of the curing treatment; and

S2013: performing a mold release treatment.

In the step S2011, a mold having an integral structure of the inner cores and the flexible substrate may be prepared in advance, which is preferably a silicone mold, and a mixed solution of PDMS and a hardening agent is then placed in the mold to be subjected to a curing treatment. The mixed solution of the PDMS and the hardening agent was prepared as follows: after uniformly mixing a main agent of PDMS and a hardening agent in a mass ratio of 10:1, bubbles of the mixed solution were floated to the surface and cracked by means of evacuation, and then the mixed solution was placed and baked in an oven of 120 degrees Celsius for about one hour. The proportions of the main agent of PDMS and the hardening agent, the bake temperature and time for baking may be determined based on different requirements of hardness of the finally prepared PDMS product. The curing treatment may be performed by heating so as to accelerate the curing speed.

In the manufacturing method provided in the embodiment of the present disclosure, the step S202 may comprise (not shown in the figures): coating a solution of metallic carbon nanotubes on an outer side of each of the inner cores to form a conductive thin film using a dip-coating method, thereby forming the protruding structures.

In an optional embodiment, in the step S202, the inner cores may be coated with a high-purity metallic carbon nanotube solution using a dip-coating method in which a dip-coating speed is optionally 0.01 mm/s to 0.005 mm/s and the dip-coating may be optionally performed 3 to 10 times. In order to ensure that the metallic carbon nanotubes form a thin film, the surface of the inner core needs to be coated with 30 to 80 carbon nanotubes per square microns. In an optional embodiment, the formed protruding structure has a radius of about 0.5 to 2.0 mm, for example, about 1.0 mm or 1.5 mm.

In the step S203, electrode lead-out terminals and electrode lead-out wires were manufactured using a spraying process or an inkjet printing process by means of a mask plate having combined patterns of the electrode lead-out terminals and the electrode lead-out wires.

In an optional embodiment, prior to coating the solution of the metallic carbon nanotubes on the outer side of the inner cores using the dip-coating method, the manufacturing method further comprises (not shown in the figures) subjecting the solution of the metallic carbon nanotubes to acid treatment.

In this way, it is possible to improve the electrical conductivity of the metallic carbon nanotube. In order to make the conductive thin film, the lead-out terminals and the lead-out wires have relatively high electrical conductivity, it is preferable to use single-walled carbon nanotube (M-SWCNT) solution with a high ratio of metallic carbon nanotube. Moreover, the acid treatment may also reduce the resistance of the thin film having light transmittance of about 80% from 500 Ω/sq to 70 Ω/sq. In a further optional embodiment, a solution of carbon nanotubes dispersed in a mixed solution of an aqueous solution of 1-propanol and perfluorosulfonated resin is used to be coated on a substrate to form a film. Due to the influence of P-type doping, the electrical conductivity may be further improved. Upon further optimization, a thin film having a resistivity of about 100 Ω/sq and light transmittance of about 80% can be obtained and the requirement of mass production can be met according to the method of the present disclosure.

Method 2

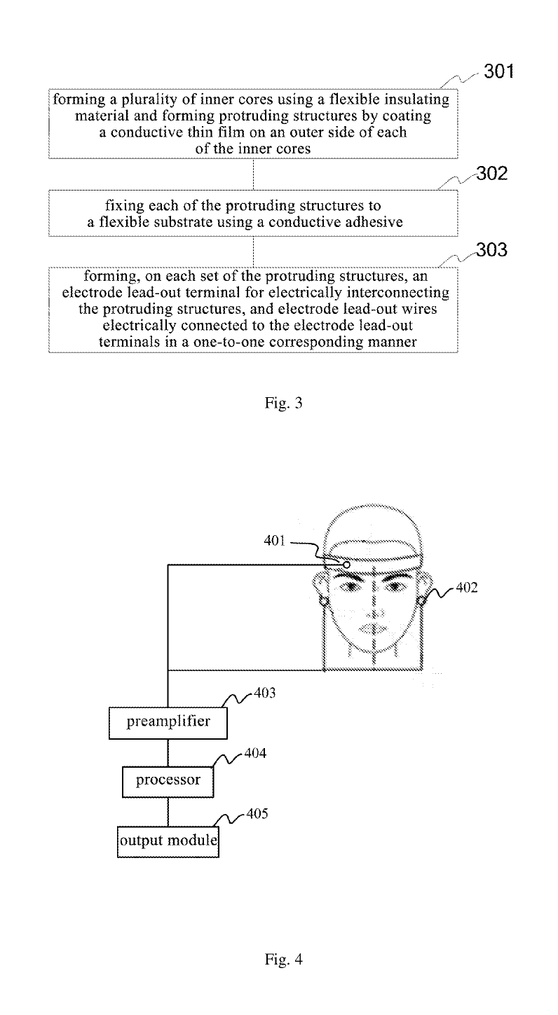

Based on the same inventive concept, this embodiment of the present disclosure provides a method of manufacturing the above-mentioned dry electrode for a bio-electromagnetic wave detecting device. As shown in FIG. 3, the method may comprise the following steps:

S301: forming a plurality of inner cores using a flexible insulating material and forming protruding structures by coating a conductive thin film on an outer side of each of the inner cores;

S302: fixing each of the protruding structures to a flexible substrate using a conductive adhesive;

S303: forming, on each set of the protruding structures, an electrode lead-out terminal for electrically interconnecting the protruding structures, and electrode lead-out wires electrically connected to the electrode lead-out terminals in a one-to-one corresponding manner.

By forming the protruding structures first and then fixing each of the protruding structures to the flexible substrate, the process of coating the conductive thin film on the outer sides of the inner cores may be simpler.

In the manufacturing method provided in this embodiment of the present disclosure, the step S301 may comprise (not shown in the figure):

S3011: forming the plurality of the inner cores using polydimethylsiloxane;

S3012: immersing each of the inner cores in a solution of metallic carbon nanotubes for a period of time to form the conductive thin film coated on the outer side of each of the inner cores, thereby forming the protruding structures.

In the step S3012, the plurality of the inner cores was immersed in the solution of metallic carbon nanotubes. In an optional embodiment, the plurality of the inner cores was immersed in a high-purity metallic carbon nanotube solution. In an optional embodiment, the immersing time was set to 4 to 8 hours. This time may be selected as actually needed, and is not limited thereto. Moreover, in order to ensure that the metallic carbon nanotubes form a thin film, the surface of the inner core is coated with 30 to 80 carbon nanotubes per square microns. In an optional embodiment, the formed protruding structure has a radius of about 0.5 to 2.0 mm, for example, about 1.0 mm or 1.5 mm.

In the step S303, electrode lead-out terminals and electrode lead-out wires were manufactured using a spraying process or an inkjet printing process by means of a mask plate having combined patterns of the electrode lead-out terminals and the electrode lead-out wires. It is also possible to first make the electrode lead-out terminals and the electrode lead-out wires on the flexible substrate and then fix the protruding structures to corresponding positions, after the step S301. The order of the steps S302 and S303 is not defined here. The spraying process in which the metallic carbon nanotube solution was directly sprayed onto the substrate exhibits a high film-forming rate and is applicable to the preparation of a large-area thin film, and the thickness of the thin film may be controlled by controlling the flow, time of spraying and the concentration of the dispersion solution.

In an optional embodiment, prior to immersing each of the inner cores in the solution of metallic carbon nanotubes for the period of time, the method may further comprise (not shown in the figures) subjecting the metallic carbon nanotube solution to acid treatment.

In this way, it is possible to improve the electrical conductivity of the metallic carbon nanotube. In order to make the conductive thin film, the lead-out terminals and the lead-out wires have relatively high electrical conductivity, it is preferable to use a metallic single-walled carbon nanotube (M-SWCNT) solution with a high ratio of metallic carbon nanotube.

Based on the same inventive concept, the present disclosure further provides in an embodiment a bio-electromagnetic wave detecting device. As shown in FIG. 4, the bio-electromagnetic wave detecting device comprises a working electrode 401 for detecting a potential at a position to be detected in an organism and a reference electrode 402; a preamplifier 403 connected to both the working electrode 401 and the reference electrode 402; and a processor 404 connected to the a preamplifier 403 and an output module 405 connected to the processor 404, wherein the working electrode and/or the reference electrode are the dry electrode for a bio-electromagnetic wave detecting device as described above.

A further embodiment of the present disclosure provides a sensor element for the bio-electromagnetic wave detecting device, which comprises a working electrode 401 for detecting a potential at a position to be detected in an organism and a reference electrode 402, as shown in FIG. 4.

Since the bio-electromagnetic wave detecting device solves the above-mentioned problem based on a similar principle as the dry electrode mentioned above, for embodiments of the bio-electromagnetic wave detecting device, you may refer to those of the dry electrode, and the embodiments will not be repeated here.

The bio-electromagnetic wave detecting device provided in the embodiment of the present disclosure may be a brainwave detecting device or an electrocardiogram detecting device.

The bio-electromagnetic wave detecting device will be illustrated by taking the brainwave detecting device for example.

The principle of the brainwave detecting device is as follows:

The activity of human brain neurons can generate persistent rhythmic potential change (i.e., brainwave). By arranging electrodes to make contact with the scalp, it is able to record the potential change generated by the brain neurons. An electrode whose potential is set as zero is referred to as a reference electrode, and other electrodes are referred to as a working electrode. The reference electrode and the working electrode are respectively connected to an electroencephalograph via lead wires such that the potential difference between the working electrode and the reference electrode is amplified, and a waveform formed due to the change in the potential difference recorded in the electroencephalograph is the brainwave.

A health condition of a human body may be judged by identifying brainwaves at different positions, and features of different types of brainwaves are shown in Table 1.

TABLE 1
features of different types of brainwaves

| Types of | | | | |
|---|---|---|---|---|
| brainwaves | Detected sites | frequency | amplitude | features |
| α waves | occipital | 8-13 Hz | 20-100 μV | Occur while awake, quiet or |
| | bone | | | closing eyes |
| β waves | frontal or | 18-30 Hz | 5-20 μV | Occur when the brain is a |
| | temporal part | | | little excited |
| θ waves | | 4-7 Hz | 10-50 μV | Occur when sleepy or in the |
| | | | | inhibition state of central |
| | | | | nervous system |
| δ waves | temporal and | 1-3.5 Hz | 20-200 μV | Occur in sleep, deep |
| | parietal lobes | | | anesthesia, hypoxia or when |
| | | | | an organic disease appears in |
| | | | | the brain |

In the bio-electromagnetic wave detecting device provided in the embodiment of the present disclosure, the brain wave detecting device adopts a method using unipolar leads. The method using unipolar leads is simple, convenient to operate and easy to realize the commercialization.

With reference to FIG. 4, the method using unipolar leads is based on the following principle: the working electrode 401 is placed on the scalp (in FIG. 4, the working electrode 401 is placed on the forehead), the reference electrode 402 is placed on the auricular lobule, and they are respectively connected to two input terminals of a preamplifier 403 through a lead selection switch. The working electrode 401 and the reference electrode 402 may be placed according to the standard international 10-20 electrode system (including 19 electrodes), and also can be placed using the international standard in which the number of the electrodes is expanded to 70. Or, the working electrode 401 and the reference electrode 402 are designed as desired. The positions of the working electrode 401 and the reference electrode 402 are not defined here. The preamplifier 403 amplifies the potential difference between the working electrode 401 and the reference electrode 402, a processor 404 performs other processing of the data and finally the output module 405 outputs a brainwave.

The electrocardiogram detecting device is operated based on a similar principle as the brainwave detecting device, and also adopts electrodes to detect the change in the potential. The potential difference is amplified by a preamplifier and this prevents change of data in the transmission process, other processing of the data is performed by a processor and finally an electrocardiogram is output by an output module. In the bio-electromagnetic wave detecting device, the dry electrode provided in the embodiments of the present disclosure is used, so it is possible to reduce the contact resistance generated when the dry electrode is in contact with the skin or scalp, thereby reducing the signal interference and increasing the accuracy of the detection data.

According to the embodiments of the present disclosure, the dry electrode for a bio-electromagnetic wave detecting device, its manufacturing method and the sensor element and the bio-electromagnetic wave detecting device comprising the dry electrode are provided. In the dry electrode, the protruding structure comprises the inner core made of the flexible insulating material and the conductive thin film coated on the outer side of the inner core. Thus, when the dry electrode in contact with the skin, a small contact resistance is generated, thus making it possible to reduce the signal interference and increase the accuracy of the detection data. Moreover, since both the flexible substrate and the inner cores are made of the flexible material, and the conductive thin film coated on the outer side of the inner core is very thin, the dry electrode is flexible and when the dry electrode is in contact with the skin, no discomfort sensation will be felt even in close contact and the comfort is increased during the detection of the bio-electromagnetic wave. The dry electrode may be applied to various bio-electromagnetic wave detecting devices, for example, a brainwave detecting device or an electrocardiogram detecting device. Further, it may also be applied to non-medical fields such as commercial brainwave toy products, for example brainwave smart wearable products.

Obviously, a person skilled in the art may make modifications and variations to the present disclosure without departing from the sprit and scope of the present disclosure. These modifications and variations are intended to be included in the present disclosure if they fall within the scope of the claims of the present disclosure and equivalents thereof.

What is claimed is:

1. A dry electrode for a bio-electromagnetic wave detecting device, comprising: a flexible substrate, at least one set of protruding structures arranged on the flexible substrate, an electrode lead-out terminal for electrically interconnecting the protruding structures in each set of the protruding structures, and electrode lead-out wires electrically connected to the electrode lead-out terminals in a one-to-one corresponding manner,
wherein the protruding structure comprises an inner core made of a flexible insulating material, and a conductive thin film coated on an outer side of the inner core.

2. The dry electrode according to claim 1, wherein the conductive thin films, the electrode lead-out terminals and the electrode lead-out wires are made of a metallic carbon nanotube material.

3. The dry electrode according to claim 1, wherein the flexible substrate and the inner cores are made of polydimethylsiloxane.

4. The dry electrode according to claim 3, wherein the inner cores and the flexible substrate are an integrally-formed structure.

5. The dry electrode according to claim 3, wherein the inner cores are fixed to the flexible substrate using a conductive adhesive.

6. A method of manufacturing the dry electrode according to claim 1, comprising steps of:
providing a flexible substrate and inner cores arranged on the flexible substrate;
forming protruding structures by coating a conductive thin film on an outer side of each of the inner cores; and
forming, on each set of the protruding structures, an electrode lead-out terminal for electrically interconnecting the protruding structures, and electrode lead-out wires electrically connected to the electrode lead-out terminals in a one-to-one corresponding manner.

7. The manufacturing method according to claim 6, wherein the flexible substrate and the inner cores arranged on the flexible substrate are formed integrally.

8. The manufacturing method according to claim 7, wherein the step of integrally forming the flexible substrate and the inner cores arranged on the flexible substrate comprises:
placing a mixed solution of polydimethylsiloxane and a hardening agent in a mold and subjecting it to a curing treatment;
forming an integrally-formed structure of the inner cores and the flexible substrate after the completion of the curing treatment; and
performing a mold release treatment.

9. The manufacturing method according to claim 6, wherein the step of forming the protruding structure by coating the conductive thin film on the outer side of each of the inner cores comprises:
coating a solution of metallic carbon nanotubes on the outer side of each of the inner cores to form the conductive thin film using a dip-coating method, thereby forming the protruding structures.

10. The manufacturing method according to claim 7, wherein the step of forming the protruding structures by coating the conductive thin film on the outer side of each of the inner core comprises:
coating a solution of metallic carbon nanotubes on the outer side of each of the inner cores to form the conductive thin film using a dip-coating method, thereby forming the protruding structures.

11. The manufacturing method according to claim 9, wherein prior to coating the solution of the metallic carbon nanotubes on the outer side of each of the inner cores using the dip-coating method, the manufacturing method further comprises subjecting the solution of the metallic carbon nanotubes to acid treatment.

12. The manufacturing method according to claim 10, wherein prior to coating the solution of the metallic carbon nanotubes on the outer side of each of the inner cores using the dip-coating method, the manufacturing method further comprises subjecting the solution of the metallic carbon nanotubes to acid treatment.

13. A method of manufacturing the dry electrode according to claim 5, comprising steps of:
forming a plurality of inner cores using a flexible insulating material and forming protruding structures by coating a conductive thin film on an outer side of each of the inner cores;
fixing each of the protruding structures to a flexible substrate using a conductive adhesive; and
forming, on each set of the protruding structures, an electrode lead-out terminal for electrically interconnecting the protruding structures, and electrode lead-out wires electrically connected to the electrode lead-out terminals in a one-to-one corresponding manner.

14. The manufacturing method according to claim 13, wherein the step of forming the plurality of the inner cores using the flexible insulating material and forming the protruding structures by coating the conductive thin film on the outer side of each of the inner cores comprises:
forming the plurality of the inner cores using polydimethylsiloxane; and
immersing each of the inner cores in a solution of metallic carbon nanotubes for a period of time to form the conductive thin film coated on the outer side of each of the inner cores, thereby forming the protruding structures.

15. The manufacturing method according to claim 14, wherein prior to immersing each of the inner cores in the solution of the metallic carbon nanotubes for the period of time, the method further comprises subjecting the solution of the metallic carbon nanotubes to acid treatment.

16. A sensor element for a bio-electromagnetic wave detecting device, comprising a working electrode for detecting a potential at a position to be detected in an organism and a reference electrode, wherein the working electrode and/or the reference electrode are the dry electrode according to claim 1.

17. A bio-electromagnetic wave detecting device, comprising: a working electrode for detecting a potential at a position to be detected in an organism and a reference electrode; a preamplifier connected to the working electrode and the reference electrode; and a processor connected to the preamplifier and an output module connected to the processor,
wherein the working electrode and/or the reference electrode are the dry electrode according to claim 1.

18. The bio-electromagnetic wave detecting device according to claim 16, wherein the conductive thin film, the electrode lead-out terminals and the electrode lead-out wires are made of a metallic carbon nanotube material.

19. The bio-electromagnetic wave detecting device according to claim 17, wherein the flexible substrate and the inner cores are made of polydimethylsiloxane.

20. The bio-electromagnetic wave detecting device according to claim 17, wherein the bio-electromagnetic wave detecting device is a brainwave detecting device or an electrocardiogram detecting device.
